(19) United States
(12) Patent Application Publication
(10) Pub. No.: US 2023/0233834 A1
(43) Pub. Date: Jul. 27, 2023

(54) COLLAPSING AND EXPANDING STRUCTURES WITH SHAPE MEMORY MATERIALS AT MULTIPLE TEMPERATURES

(71) Applicants: Theodosios Alexander, Venice, FL (US); Martin T. Rothman, Bodega Bay, CA (US)

(72) Inventors: Theodosios Alexander, Venice, FL (US); Martin T. Rothman, Bodega Bay, CA (US)

(21) Appl. No.: 18/055,332

(22) Filed: Nov. 14, 2022

Related U.S. Application Data
(60) Provisional application No. 63/279,924, filed on Nov. 16, 2021.

(51) Int. Cl. A61M 60/13; A61F 2/24; A61F 2/844; A61M 60/178; A61M 60/237; A61M 60/405; A61M 60/808; A61M 60/419

(52) U.S. Cl. CPC A61M 60/13; A61F 2/2412; A61F 2/844; A61M 60/178; A61M 60/237; A61M 60/405; A61M 60/808; A61M 60/419; A61M 2205/0266; A61F 2210/0028; A61F 2210/0042

(57) ABSTRACT

Shape memory alloys are used in aerospace structures, orthodontics, cardiovascular prosthetic devices, sensors and controllers, and many other engineering, technology, science, and other fields. The methods are described in the case of a temporary heart assist pump to illustrate the concepts, but the method applies to many other fields. The properties of shape memory alloys are used to fold or collapse and implant in the human body a device without breaking the device as it reaches body temperature or without reaching permanent plastic deformation. The properties of nitinol are also used to describe intended explantation of the device, at body temperature, from the body without breaking it. Such planned explantation may be needed in cases where the device is designed for temporary use, such as mechanical circulatory support devices intended for temporary use and then removal of all components of the device from the body. The same method can be used for devices that have not been initially designed for removal, such as stents or valves, that must later be explanted for reasons unanticipated when they were installed. The methods ensure that the devices stay within stress-strain-temperature conditions so they remain elastic, or under the upper stress plateau, or remain plastic, but always under the breaking strain, of shape memory alloys at: room or environmental conditions; cooler than environmental conditions; and at a higher temperature, or body temperature. The methods described may also be applied to other industrial applications, where shape memory alloys may be installed and removed at different temperatures. Applications in other industries, include aerospace, civil structures, mechanical structures are contemplated.

### INCORPORATION BY REFERENCE

This application claims priority benefit of U.S. Provisional Patent Application No. 63/279,924 filed Nov. 16, 2021, which is incorporated herein by reference in its entirety for all purposes. Any and all applications related thereto by way of priority thereto or therefrom are hereby incorporated by reference in their entirety. Systems and methods as disclosed herein can include any combination of features disclosed, for example, in PCT/US2019/025667 filed Apr. 3, 2019, PCT/US2020/039978 filed Jun. 26, 2020, U.S. Provisional Patent Application No. 63/279,826 filed Nov. 16, 2021, a nonprovisional utility patent application entitled COLLAPSING MECHANICAL CIRCULATORY SUPPORT DEVICE FOR TEMPORARY USE, filed on the same day herewith, which are hereby incorporated by reference in their entireties.

### BACKGROUND

### Field

Some embodiments of the present invention relate to a mechanical circulatory support device, for assisting or replacing native heart function in cases of congestive heart failure. Some embodiments also relate to percutaneously implantable cardiovascular support and percutaneously implantable temporary mechanical circulatory support device. The methods have far-reaching implications for implantation and removal of implantable devices. The methods may be applied to other industrial applications, where shape memory alloys may be installed and removed at different temperatures.

### SUMMARY

Shape memory alloys are used in aerospace structures, orthodontics, cardiovascular prosthetic devices, sensors and controllers, and many other engineering, technology, science, and other fields. Solar panels used in space may be folded and unfolded at two different temperatures on ground, and folded and unfolded at a third colder temperature in space. Orthotic, orthodontic and cardiovascular devices experience operating room ambient temperature about 20 degree C., may be folded at 0 degree C., and unfolded and folded at body temperature about 37 degree C. Other literature in these fields describes the use of differences in stress-strain curves in shape-memory alloys with transition from martensite to austenite at two different temperatures. The subject of this disclosure is when three or more different temperatures are used in the practical application. This disclosure expands the field of application from two to three or more different temperatures. The use of the method is described in the case of a temporary heart assist pump to illustrate the concepts, but the method applies to many other fields.

It is an object of the invention to provide a device that can be installed and removed with less risk to the patient.

In some embodiments, a method to use the temperature versus stress versus strain properties of shape-memory alloys to ensure that the device stays in the elastic regime in at least three different temperatures used for different temperature conditions is provided.

In some embodiments, the temperatures are selected from a collapsed cold temperature, an expanded hot temperature, a collapsed hot temperature, and an environmental temperature.

In some embodiments, a method to use the temperature versus stress versus strain properties of shape memory alloys to ensure that the device, when deformed, stays in the elastic regime in at least three different temperatures used for different temperature conditions is provided.

In some embodiments, a method to use the temperature versus stress versus strain properties of shape-memory alloys to ensure that the device, when deformed, stays below a targeted permanent strain level when deformed in any of at least three different temperatures used for different temperature conditions is provided.

In some embodiments, a method to use the temperature versus stress versus strain properties of shape-memory alloys to ensure that the device, when deformed, stays above the elastic, but in the plastic regime below fracture, in at least three different temperatures used for different temperature conditions is provided.

In some embodiments, a method to use the temperature versus stress versus strain properties of shape-memory alloys to ensure that the device, when deformed, stays anywhere below the fracture point in at least three different temperatures used for different temperature conditions is provided.

In some embodiments, a method to use the temperature versus stress versus strain properties of shape-memory alloys to ensure that the device is able to recover its shape without deformation in at least three different temperatures used for different temperature conditions is provided.

In some embodiments, the temperatures are selected from a collapsed cold temperature, an expanded hot temperature, a collapsed hot temperature, and an environmental temperature.

In some embodiments, a method to use the temperature versus stress versus strain properties of shape-memory alloys to ensure that the device stays below fracture limits in at least three different temperatures used for three different conditions is provided.

In some embodiments, the temperatures are selected from a collapsed cold temperature, an expanded hot temperature, a collapsed hot temperature, and an environmental temperature.

In some embodiments, a method to use the temperature versus stress versus strain properties of shape-memory alloys to ensure that implanted devices stay in the shape-recovering regime at temperatures between T2 and T8, thus facilitating removal of the implanted devices after use is provided.

In some embodiments, T2 and T8 are environmental and body temperature. In some embodiments, T2 and T8 are related to aerospace applications. In some embodiments, T2 and T8 are related to aerospace applications temperatures. In some embodiments, T2 and T8 are higher and lower than environmental temperatures. In some embodiments, the method includes facilitating collapse, implantation, and removal after use, at two or more temperatures different than environmental. In some embodiments, the method includes facilitating collapse, implantation, and removal after use, at three or more temperatures, wherein the third temperature is environmental. In some embodiments, T2 and T8 are a cold collapsed temperature and a body temperature.

In some embodiments, a method to use the temperature versus stress versus strain properties of shape-memory alloys to ensure that implanted devices stay within elastic deformation limits at temperatures between the highest and lowest of three temperatures is provided.

In some embodiments, highest and lowest of three temperatures are higher and lower than environmental temperatures. In some embodiments, the method includes facilitating collapse, implantation, and removal after use, at two or more temperatures different than environmental.

In some embodiments, a method to use the temperature versus stress versus strain properties of shape-memory alloys to ensure that implanted devices stay below the fracture strain at temperatures between the maximum and minimum of three temperatures is provided.

In some embodiments, the maximum and minimum of three temperatures are higher and lower than environmental temperatures. In some embodiments, the method includes facilitating collapse, implantation, and removal after use, at two or more temperatures different than environmental. In some embodiments, the method includes facilitating collapse, implantation, and removal after use, at three or more temperatures.

In some embodiments, a method to use the temperature versus stress versus strain properties of shape-memory alloys to ensure that, after implantation, highly-stressed portions of the devices deform while staying in the elastic regime in the body at body temperature T8, thus facilitating explantation of the implanted devices after use is provided.

In some embodiments, a method to use the temperature versus stress versus strain properties of shape-memory alloys to ensure that, after implantation, highly-stressed portions of the devices deform without breaking (without reaching breaking stress) in the body at body temperature T8, thus facilitating removal of the implanted devices after use is provided. In some embodiments, a method to use the temperature versus stress versus strain properties of shape-memory alloys to ensure that, after implantation, highly-stressed portions of the devices deform without breaking (without reaching breaking strain) in the body at body temperature T8, thus facilitating removal of the implanted devices after use is provided.

In some embodiments, the device is initially at zero stress, zero strain, at room temperature in austenitic phase. In some embodiments, the device is cooled to below the temperature at which martensitic phase has finished forming. In some embodiments, the device is then collapsed for implantation at this temperature experiencing finite stress and strain. In some embodiments, the device is then inserted into the body where it reaches body temperature and austenitic state with positive stress and strain but below breaking stress. In some embodiments, the device is then inserted into the body where it reaches body temperature and austenitic state with positive stress and strain but below breaking strain. In some embodiments, the device is removed from the body without breaking after some period of use. In some embodiments, the device where after some period of use the device is removed without breaking from the body. In some embodiments, after implantation and removal of a constraining device at body temperature, the implanted device is returned to austenitic state and zero-stress zero strain. In some embodiments, after implantation and removal of a constraining device at body temperature, the implanted device is returned to the zero stress, positive strain. In some embodiments, after a period of use the implanted device is collapsed again at body temperature and austenitic phase without reaching breaking stress. In some embodiments, after a period of use the implanted device is collapsed again at body temperature and austenitic phase without reaching breaking strain. In some embodiments, after some period of use the implanted device is removed without breaking inside the body. In some embodiments, the collapsing components of the device reach specific dimensions of the impeller tip to inner diameter of waist after one cycle, thus optimizing this dimension for maximum efficiency. In some embodiments, the collapsing components of the device reach specific dimensions of the impeller tip to inner diameter of waist after a series of cycles, thus optimizing this dimension for maximum efficiency. In some embodiments, the collapsing components of the device reach specific dimensions of the impeller tip to inner diameter of waist after one cycle, thus optimizing this dimension for minimum hemolysis. In some embodiments, the collapsing components of the device reach specific dimensions of the impeller tip to inner diameter of waist after a series of cycles, thus optimizing this dimension for minimum hemolysis. In some embodiments, the physical size or geometry of the bending components has been optimized with finite element calculations to remain below the upper stress plateau at body temperature. In some embodiments, the physical size or geometry of the bending components has been optimized with finite element calculations to remain below the breaking strain point at body temperature. In some embodiments, the physical size or geometry of the bending components has been optimized with theoretical calculations to remain below the upper stress plateau at body temperature. In some embodiments, the physical size or geometry of the bending components has been optimized with theoretical calculations to remain below permanent deformation at body temperature. In some embodiments, the physical size or geometry of the bending components has been optimized with theoretical calculations to remain below permanent deformation at targeted removal temperature. In some embodiments, the physical size or geometry of the bending components has been optimized with theoretical calculations to remain below permanent deformation stress. In some embodiments, the physical size or geometry of the bending components has been optimized with theoretical calculations to remain below permanent deformation strain. In some embodiments, the physical size or geometry of the bending components has been optimized with finite theoretical calculations to remain below the breaking strain point at body temperature. In some embodiments, the physical size or geometry of the bending components has been optimized with manufactured prototype experiments to remain below the upper stress plateau at body temperature. In some embodiments, the physical size or geometry of the bending components has been optimized with manufactured prototype experiments to remain below permanent deformation at body temperature. In some embodiments, the physical size or geometry of the bending components has been optimized with manufactured prototype experiments to remain below permanent deformation at targeted removal temperature. In some embodiments, the physical size or geometry of the bending components has been optimized with manufactured prototype experiments to remain below permanent deformation stress. In some embodiments, the physical size or geometry of the bending components has been optimized with manufactured prototype experiments to remain below the breaking strain point at body temperature. In some embodiments, the geometric shape is optimized by removing alloy material. In some embodiments, the geometric shape is optimized by adding alloy material. In some embodiments, the geometric shape is optimized by removing alloy material or adding alloy material, thus implementing changes in the stiffness of highly-stressed bending portions to facilitate bending into the desirable state while keeping the stress below the upper stress plateau. In some embodiments, the geometric shape is optimized by removing alloy material, or adding alloy material, thus implementing changes in the stiffness of highly-stressed twisting portions to facilitate twisting into the desirable state while keeping the stress below the upper stress plateau. In some embodiments, the geometric shape is optimized by removing alloy material, or adding alloy material, thus implementing changes in the stiffness of highly-stressed bending portions to facilitate bending into the desirable state while keeping the strain below the breaking strain. In some embodiments, the geometric shape is optimized by removing alloy material, or adding alloy material, thus implementing changes in the stiffness of highly-stressed twisting portions to facilitate twisting into the desirable state while keeping the strain below the breaking strain. In some embodiments, the geometric shape of the medical device is optimized to allow inserting guiding shapes of a biocompatible material to collapse the device, thus implementing changes in the stiffness of highly-stressed bending portions to facilitate bending into the desirable state while keeping the stress below the upper stress plateau. In some embodiments, the geometric shape of the medical device is optimized to allow inserting guiding shapes of a biocompatible material such as a catheter to collapse the device, thus implementing changes in the strain of highly-stressed twisting portions to facilitate twisting into the desirable state while keeping the stress below the upper stress plateau. In some embodiments, the geometric shape of the medical device is optimized to allow inserting guiding shapes of a biocompatible material such as a catheter to collapse the device, thus implementing changes in the stiffness of highly-stressed twisting portions to facilitate twisting into the desirable state while keeping the stress below the upper stress plateau. In some embodiments, the geometric shape of the medical device is optimized to allow inserting guiding shapes of a biocompatible material to facilitate collapsing the device into a catheter, thus implementing changes in the strain of highly-stressed bending portions to facilitate bending of the medical device into the desirable state while keeping the strain below the breaking strain. In some embodiments, the geometric shape of the medical device is optimized to allow inserting guiding shapes of a biocompatible material to facilitate collapsing the device into a catheter, thus implementing changes in the strain of highly-stressed bending portions to facilitate bending of the medical device into the desirable state while keeping the strain within recoverable elastic limits. In some embodiments, the geometric shape of the medical device is optimized to allow inserting guiding shapes of a biocompatible material such as a catheter to collapse the device, thus implementing changes in the stiffness of highly-stressed bending portions to facilitate bending of the medical device into the desirable state while keeping the strain below the breaking strain. In some embodiments, the geometric shape of the medical device is optimized to allow inserting guiding shapes of a biocompatible material such as supporting structures 1780 allow collapsing in a catheter, thus implementing changes in the stiffness of highly-stressed twisting portions to facilitate twisting of the medical device into the desirable state while keeping the strain below the breaking strain. In some embodiments, the geometric shape of the medical device is optimized to allow inserting guiding shapes of a biocompatible material such as a catheter to collapse the device, thus implementing changes in the stiffness of highly-stressed twisting portions to facilitate twisting of the medical device into the desirable state while keeping the strain below the breaking strain.

In some embodiments, wherein the method is applied to the components of collapsible heart assist pumps, prosthetic heart valves, or stents. In some embodiments, wherein the method is applied to different industries and uses. In some embodiments, the environmental temperature is room temperature. In some embodiments, the environmental temperature is body temperature. In some embodiments, the environmental temperature is ice bath temperature. In some embodiments, the method is limited to the biomedical field. In some embodiments, the method is limited to the mechanical circulatory support field. In some embodiments, a curvature controller comprises a varying radius distribution to accommodate stress or strain levels below a desired point along the length of a blade-hub interconnect of a blade of the implanted device. In some embodiments, a curvature controller comprises a changing radius to control the rate of distribution of stress, or strain, along the bending shape of a blade-hub interconnect of a blade of the implanted device. In some embodiments, a curvature controller comprises a changing radius to keep the combined bending and torsional stresses below a target level. In some embodiments, a curvature controller comprises a changing radius to keep the combined bending and torsional strain below a target level. In some embodiments, a curvature controller comprises a changing radius to keep the combined multi-dimensional strain below a target level. In some embodiments, a curvature controller comprises a changing radius to keep the resultant strain below a target level for deformation control. In some embodiments, a curvature controller comprises a changing radius to keep the combined bending and torsional stresses below a target level. In some embodiments, a curvature controller comprises a changing radius to keep the resultant combined strain below a target level for deformation control.

### BRIEF DESCRIPTION OF THE DRAWINGS

Embodiments of the invention will now be described, by way of example only, with reference to the accompanying drawings in which corresponding reference symbols indicate corresponding parts, and in which:

FIGS. 1-20 illustrate various features.

### DETAILED DESCRIPTION

The properties of shape memory alloys are used to fold or collapse a device. The device can be used to implant in the human body. The properties of shape memory alloys are used to fold or collapse the device without breaking the device as the device reaches body temperature. In some embodiments, the shape memory alloys comprises nitinol.

The properties of nitinol are also used to describe intended explantation or removal of the device, at body temperature, from the vasculature without breaking the device. The planned removal may be needed in cases where the device is designed for temporary use, such as mechanical circulatory support devices intended for temporary use and then removal of all components of the device from the body. The same method can be used for devices that have not been initially designed for removal, such as stents or valves, that must later be explanted for reasons unanticipated when they were installed.

The methods ensure that the devices stay within stress-strain-temperature conditions so they remain elastic, or under the upper stress plateau, or remain plastic, but always under the breaking strain, of shape memory alloys at the following temperatures: environmental conditions, cooler than environmental conditions, and at a higher than environmental conditions. The methods ensure that the devices stay within stress-strain-temperature conditions so they remain elastic, or under the upper stress plateau, or remain plastic, but always under the breaking strain, of shape memory alloys at the following temperatures: room temperature, cooler than room temperature, and higher than room temperature. The methods ensure that the devices stay within stress-strain-temperature conditions so they remain elastic, or under the upper stress plateau, or remain plastic, but always under the breaking strain, of shape memory alloys at the following temperatures: cooler than body temperature such as room temperature and at body temperature. The environmental conditions can be room temperature. The environmental conditions can be body temperature. The environmental conditions can be ice bath temperature. The environmental conditions can be conditions in which the device reaches 0 degrees Celsius. The environmental conditions can be temperatures achieved with the use of cold sprays. The environmental conditions can be conditions in which the device reaches temperatures below 0 degrees Celsius. The environmental conditions can be conditions in which the device reaches temperatures between −10 degrees Celsius and −20 degrees Celsius.

In aerospace applications, the method may be used to fold and unfold solar panels in space, to modify the shape of airfoils and wings to achieve variations in lift and drag in airplanes, to modify the shape of flying objects in order to control observable reflections of optical, acoustic, electrical or magnetic reflections for stealth operations, among other uses. In civil engineering applications, the method may be used to control deflections of structures at different ambient temperatures. In automation and controls, the method may be used to modulate signal amplitude and control function with dependence on ambient or operating temperature. Without loss of generality, the method is described with the example of a heart-assist pump implanted for temporary use, then removed at a third operating temperature without breaking the device inside the human body. The biomedical field is just now beginning to realize the need to fold and explant Nitinol heart valves to implant a new one, for instance after use of the first valve implanted for some years in young people. Similarly but less frequently, implanted stents may need to be explanted. Such requirements for removal without breakage at a different temperature are addressed herein. The methods described may also be applied to other industrial applications, where shape memory alloys may be installed and removed at different temperatures. The methods can provide coverage for biomedical field. The methods can provide coverage for industrial processes in different fields. The methods can provide coverage for stents or valves. The methods can provide coverage for mechanical circulatory supports.

Without loss of generality, as an example of where the method may be used, shape memory alloys can be used in cardiovascular stents, prosthetic heart valves, and removable heart-assist pumps for temporary use. The method has application in many industrial fields where the shape memory alloys need to be collapsed in different temperatures.

A small portion of cardiovascular stents may need to be explanted because they may get infected, stenose, rupture, migrate, or exhibit internal or external leaks not suitable for endoluminal therapy, or limb thrombosis. The methods described below can be used to ensure that the stent is flexible enough to be collapsed into a catheter for explantation without fracturing inside the patient's body.

Another portion of cardiovascular prosthetic devices, such as heart valves, have a limited life. In cases where the life expectancy of the patient exceeds the life expectancy of the valve, the valve must be replaced. In other cases, the valves exhibit regurgitation, thrombose, cause infective endocarditis, or structural valve failure, or manifest other complications so that the valves must be removed or replaced. It would be most desirable if there were ways to collapse these prosthetic valves to the minimal volume again to be captured, explanted, and replaced with a new one after some years of use, with minimally invasive procedures. The methods described herein can be used to ensure that removable portions of the heart valve, either the whole valve or the ring of leaflets of the valve, is flexible enough to be collapsed into a catheter without fracturing inside the patient's body.

In recent years there has been increased interest in miniature heart-assist pumps that are fully-removed after a period of use, which, which can be called temporary Mechanical Circulatory Support Devices (MCSD). MCSD are designed for implantation with minimally invasive surgery. As described herein, VADs have their inlet cannulated to the (usually left or infrequently right) ventricle. MCSD are implanted elsewhere in the vasculature. Permanent MCSD are MCSD with some of their components permanently implanted in the body. Temporary MCSD have all their components permanently removed after use.

Miniature heart-assist pumps may be used for smaller periods, varying from a few hours to a few weeks to a few months. Some of these miniature heart-assist pumps have foldable components, and after a period of use, it is desirable that they collapse again to the minimal volume state in order to be captured and removed. This is intended, planned, designed explantation. As these pumps are used in recurring conditions, in most instances a second pump may be used again after a period of time, similarly collapsed, deployed, used for some time, and then collapsed again for explantation. The patient may not need assistance from a blood pump until the next episode.

A removable heart assist pump is described in a nonprovisional utility patent application entitled COLLAPSING MECHANICAL CIRCULATORY SUPPORT DEVICE FOR TEMPORARY USE, filed on the same day herewith and U.S. Provisional Application No. 63/279,826, filed Nov. 16, 2021, which are incorporated by reference herein in their entirety. This corresponding application describes a method to remove many biocompatible devices, using this pump as an example. The method described herein may be used in other cases in engineering industry, where shape memory alloys may need to be collapsed and/or removed at a different temperature (T₈) than the initial temperature (T₁) or the temperature (T₂) at which the device is initially collapsed to minimum shape before implantation.

In current medical practice, heart assist valves are not designed for intentional explant, and in many cases their life exceeds the expected life of older patients. The way the valves are designed, many of them endothelialize with tissue around them after a period of use. Thus, explants of heart valves and stents have a time period after which the structure around the valve leaflets cannot be removed without surgery. However, as these heart valves are implanted in younger people, and as complications occur with a small number of patients of all ages, there is a small but growing number of cases where the valves must be explanted. There are some, but even fewer than valves cases, where stents need to be explanted.

Therefore, there exists a need for unintended explantation of heart valves and stents, and there may also be a need for future new designs of heart valves and stents where they may need to be explanted. For instance, the ring of leaflets of a heart valve described herein may be designed to be part of an inner ring that does not endothelialize, and be part of the intentionally or unintentionally expandable structure of the valve, which may or may not be attached to another part of the valve that is allowed to endothelialize.

The methods designed herein may be used for all the above medical applications, and may also be used in other cases in broader industry, whenever the shape memory alloy structure needs to be collapsed at two or more different temperatures.

Gold-cadmium (Au—Cd) shape-memory alloys (SMA), also frequently called smart materials, were discovered in the 1930s. The unique properties of Nickel-Titanium (Ni—Ti) alloys were first observed in 1960s. Today there are two basic families of shape memory alloys in use: copper-aluminium based (Cu—Al with Zn, Ni, Be etc.) and nickel titanium based (Ni—Ti with Fe, Cu, Co etc). Nitinol alloys used in biomedical applications may contain fractional percentages of Cr, Cu, Fe, Nb, Co, ppm of C and O etc. Theoretical and experimental data related to the properties, manufacturing and uses of shape memory materials are disclosed.

The materials have memory in that they have different stress-strain curves at different temperatures, so the materials can have distinct shapes at different temperatures. This is a continuously-varying relation to temperature as described herein. This is achieved by the transformation of their crystalline structure from austenite (A) at a higher temperature to martensite (M) at a lower temperature. The austenite phase has a simple cubic B2(CsCl) crystal structure. The martensite phase has a monoclinic B19 crystal structure. The transition between austenite and martensite occurs by energy exchange affected either thermally or by inducing stress.

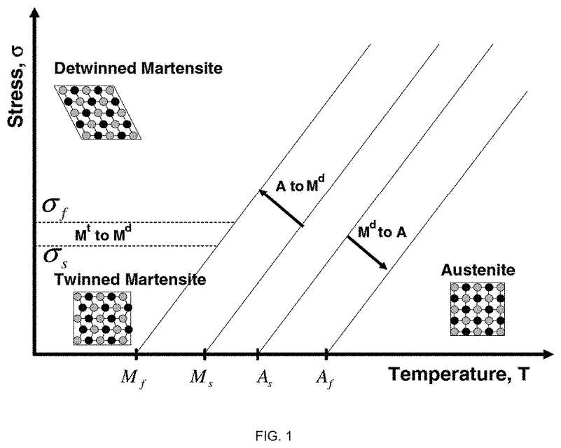

FIG. 1 is a schematic of stress-temperature diagram for a shape memory alloy. The crystallographic information and mechanical material properties at different temperatures, under constant or time varying stress-strain conditions are illustrated. At a given zero or moderate stress level, at higher temperature, the alloy is in austenitic (A) phase. As it is cooled there is a lower temperature at which the transformation to martensite (M) starts (Mₛ), and finishes at a lower temperature (M_{f}), where the material is in twinned martensite state. The reverse process is also described, where starting from twinned martensite, with increasing temperature, there is a temperature at which the austenitic transformation starts (Aₛ), and is finished (A_{f}). The stress dependence between twinned and detwinned martensite, where there is a start stress for detwinning (σₛ) and a finish temperature for detwinning (σ_{f}). There are temperature levels above which the material does not enter the Martensitic state at any stress-strain level. These effects are illustrated in FIG. 1.

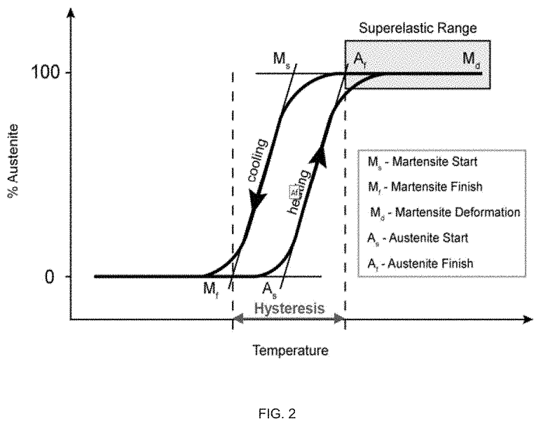

FIG. 2 illustrates the thermal hysteresis effect. Thermal hysteresis is caused by the phase transformation between martensite and austenite when the alloy is heated or cooled between A_{f} and M_{f}. This hysteresis is typically around 20-30° C. (i.e. A_{f}−M_{f}) for fully annealed Nitinol alloys, but all these theoretical values are affected by ongoing developments in alloy composition and heat treatment. Above A_{f} and up until the martensite reached deformation (Md), the alloy is in a super-elastic response range. These effects are illustrated in FIG. 2. There are variations in these levels when an SMA material undergoes cyclic stress, strain or temperature loading and unloading.

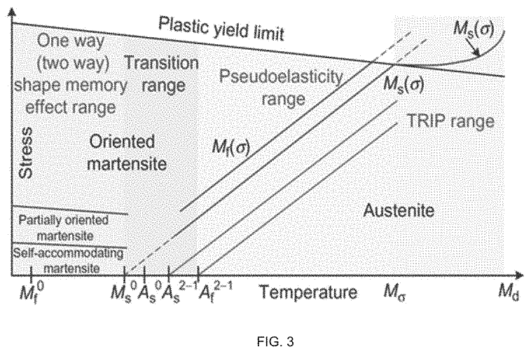

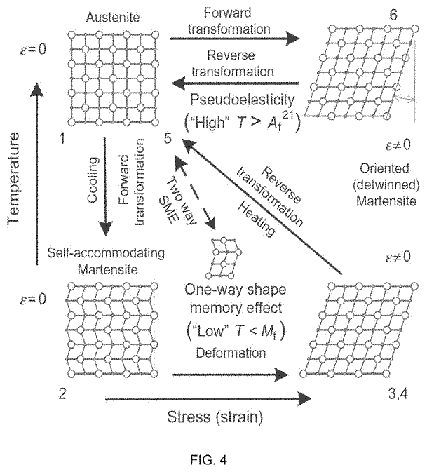

FIG. 3 illustrates shape memory alloy behavior and SME in temperature and stress coordinates. FIG. 4 illustrates a schematic illustration of SME with underlying microscopic mechanisms. Above martensite deformation temperature (Ma) the alloy is always in the austenitic phase as shown in FIG. 3. This dependence between crystalline phase, temperature, and stress and strain behavior is illustrated in FIGS. 3 and 4.

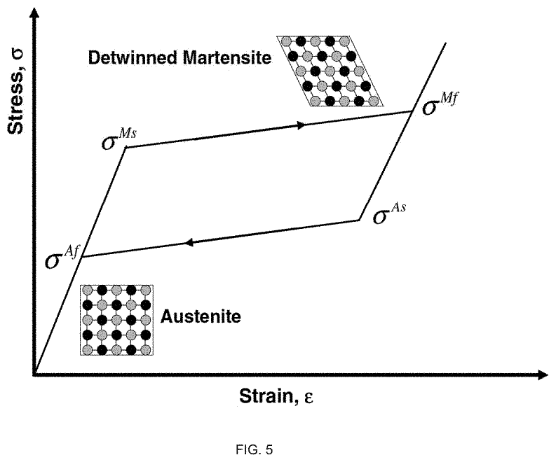

FIG. 5 illustrates schematically the pseudo-elastic stress-strain diagram for a shape memory alloy, and the theoretical pseudo-hysteresis behavior. As a result of this stress, strain and temperature dependence on changes in the crystalline behavior of the material, in certain temperature ranges, e.g. in the pseudo-elastic range (commonly called the super-elastic range), the stress-strain curve is direction-dependent and exhibits this super-elastic (pseudo-elastic) hysteresis-type behavior, caused by changes in the crystalline structure and temperature, illustrated in FIG. 5.

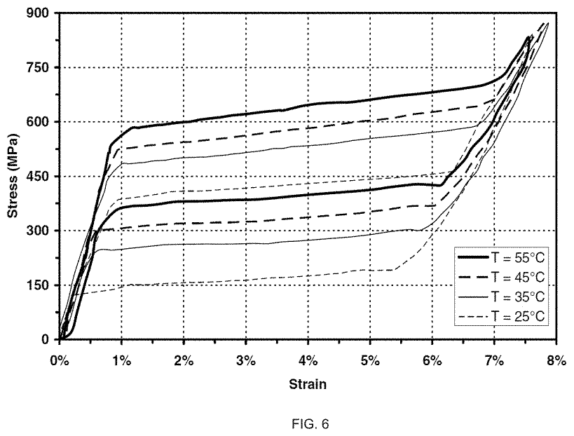

FIG. 6 illustrates the stress-strain hysteresis curve of shape memory alloys which moves to lower stress levels as temperature decreases. As temperature is decreased, the hysteresis curves are displaced to lower stress levels, as illustrated in FIG. 6. The stress-strain curves in FIGS. 5 and 6 indicate an upper stress level and a lower stress level in the direction-dependent path between stress and strain.

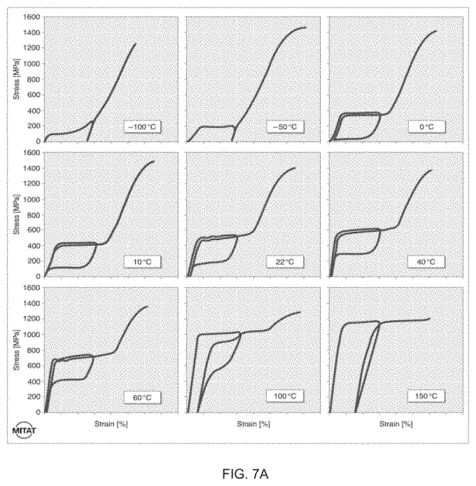

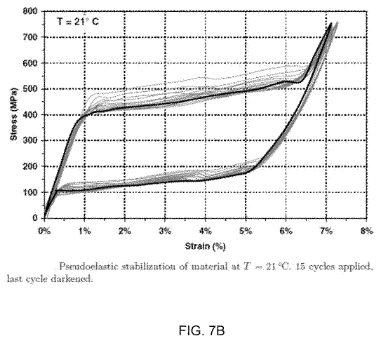

FIG. 7A illustrates the effect of test temperature on the mechanical behavior of Nitinol wire. There is a systematic increase in the upper and lower plateau stresses with increasing test temperature. Below 0° C., the structure is martensite and, above 150° C., the graph shows conventional deformation of the austenite. The intermediate temperatures indicate show classic transformational super-elasticity. Typically, the plateaus are much flatter than those in FIGS. 5 and 6, and the corresponding stress-strain segments are referred to as the Upper Stress Plateau (USP) and Lower Stress Plateau (LSP), illustrated in the tested data of FIG. 7.

The effects of chemistry composition in the alloy and heat treatment on transformation temperature can be described. The transformation temperature is very sensitive and can vary from −100 deg C. at 48.5% Ti to +100 deg C. at 51% Ti, or even over a wider range. The other constituents in the alloy also affect transformation temperature and stress-strain properties. For instance, depending on alloy composition, the pseudo-elastic hysteresis effect may be exhibited up to strains of 6% to 8% or more. The deformation processing of hot worked and cold worked Nitinol and heating durations, temperatures, and aging treatments are known to influence transformation temperatures and the shape of stress-strain curves. Thermal processing is used to tailor these properties for optimal performance. Thus, the effects of alloy atomic concentrations, and of thermal processing, for instance temperature and duration of heat treatment, affect the transformation behaviour and mechanical behaviour of Nitinol by changing the transformation temperature, the stress levels of the Upper Stress Plateau and Lower Stress Plateau, and the Ultimate stress and strain. Understanding these aspects is essential for successful application of Nitinol shape memory alloys in all fields of application.

FIG. 7B illustrates the pseudoelastic stabilization of material at temperature equal to 21 degrees C., with 15 cycles applied and the last cycle darkened. This figure indicates that repeated cycles of loading and unloading result in small changes in levels of Upper Stress Plateau, Lower Stress Plateau, and strain levels, which after a number of cycles leach terminal levels. This process of reaching terminal levels is called “training” in the nitinol field, and “reaching a limit cycle” in other engineering fields such as controls, where the “limit cycle” in these other field refers to reaching a state where these small changes from cycle to cycle stop. Thus, a limit cycle is a closed trajectory in phase space (stress-strain space here) having the property that at least one other trajectory (the final trajectory here) spirals into this cycle, after a number of repeated cycles, and after that more repetitions of the cycle repeat the same trajectory. Understanding these two process (change of dimensions after one cycle, or reaching the specific dimension trained or limit-cycle state) can be used whenever it is desired that the component reaches a specific size after one cycle, or reaches a different specific size that does not change after a number of cycles of loading and unloading. In some embodiments, these processes can be used to reach a specific target size of diameter at the inner diameter of the waist after one cycle, thus controlling the impeller-tip to diameter gap. Alternatively, the training or “limit cycle” can be used to reach a specific target size at the inner diameter of the waist after a number of cycles. This ensures a specific size of gap from impeller tip to inner diameter of the waist, thus enabling us to determine the allowable backflow (regurgitant flow), the pump hydraulic efficiency, and the level of hemolysis caused by shear of the blood in the gap between impeller tip and inner diameter of the waist.

The temperature dependence of the pseudo-elastic stress-strain curve shown in FIG. 7A has been considered as if the hysteresis-type loop moves to lower stress levels with decreasing temperature. With decreasing temperature, the Lower Stress Plateau of T1 and T8 appears to sink below the zero value on the stress axis at T2, but in practice, the stress and temperature dependent changes appear in the crystalline structure of the material as described herein in relations to FIGS. 1-4.

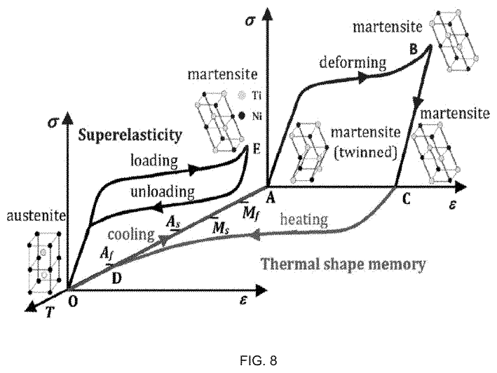

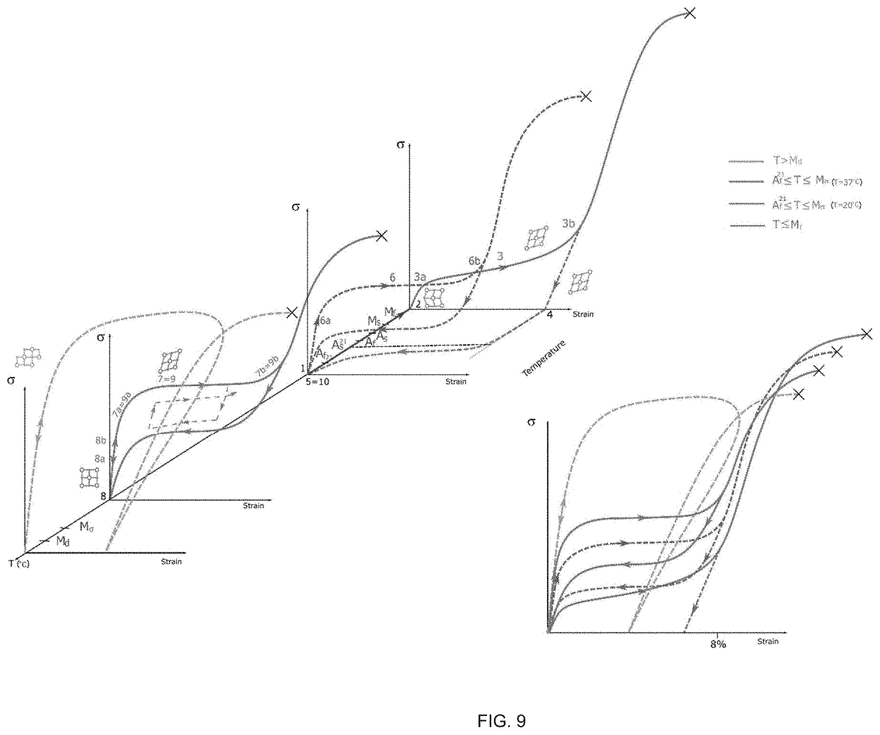

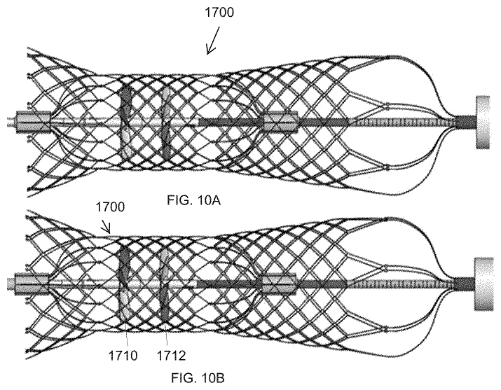

FIG. 8 illustrates the typical use of stress-strain-temperature properties of nitinol, and transformation from martensitic to twinned martensitic to austenitic state, e.g. for permanent implantation of cardiovascular stents. The implantation of shape memory alloy devices can be described in relation to FIG. 8. Point O of FIG. 8 corresponds to Point 1 and 5 of FIG. 9. FIG. 8 can explain the use of shape memory alloys and the implantation of stents. FIG. 8 is correct if the device returns to temperature TO. In FIG. 9, when devices are implanted, the device goes from colder than T1 points 3, to warmer than T1 points 7.

It is assumed that the device starts from zero-stress and zero-strain large-volume shape from temperature T_{O} above A_{f}; then cooled to T_{A} below M_{f}, still at large volume zero stress and zero strain; then compressed at temperature T_{A} (e.g. inside a catheter) to a positive stress, positive strain, smaller volume shape along the Upper Stress Plateau side of the curve to point B, and the device implanted in the body. At that point it is explained that if the catheter is removed at temperature T_{A}, and if the device has reached the Upper Stress Plateau strain levels, the device will assume a deformed intermediate volume shape at point C, with zero stress (as it is outside the catheter) and positive strain (smaller volume than it started at points O and A, but larger volume than the higher-strain point B). Then, as the device is heated, it returns to its initial shape (zero stress, zero strain, large volume shape) at point O. In some cases, stents are expanded with balloons into a desired shape while implanted. In some instances, it is assumed that from point B at temperature T_{A} it returns to point E at temperature T_{O} before the catheter is removed with the device installed in the body. With the catheter removed, the implanted device would return to point O, at zero stress, zero strain and large volume configuration. Thus the properties of nitinol have been used to implant a large-volume device (point O) by using the properties of nitinol to cool it (point A), then compress it into a small volume (point B), then implant it into the body where it returns to the large volume at point O.

“Assuming Nitinol initially is in an austenitic state at the origin point O. With no applied stress as Nitinol is cooled along path O-A below martensite finish temperature (M_{f}), complete transformation from austenite to martensite (twinned) will occur. The material is deformed through reorientation and detwinning of martensite along path A-B. Then, load releasing on path B-C will cause elastic unloading of the reoriented detwinned martensite and the material stays deformed. On heating above the austenite finish temperature (A_{f}), the material transforms from martensite to austenite and recovers the pseudo-plastic deformation ‘remembering’ its former shape. The austenitic Nitinol can be loaded along the path O-E above the austenite finish temperature (A_{f}) through a stress-induced transformation to martensitic state. A large elastic strain up to 11% can be achieved. Upon unloading along the path E-O, the material will transform back to austenitic state and the superelastic deformation will be recovered, demonstrating a hysteresis loop in the stress-strain diagram.” Y Guo, A Klink, C Fu, J Snyder. Machinability and surface integrity of Nitinol shape memory alloy. CIRP Annals—Manufacturing Technology 62 (2013) 83-86.

There is infrequent mention of the difference between room temperature in the operating room, about 20 deg C., and body temperature, typically around 36.7 or 37 deg C., or how these may affect implantation of the device. Despite the recognition of the effect of temperature M_{D} in relation to the maximum temperature of the crystalline transformation process, it is assumed that the properties will work for implantation. This means the device is considered from room temperature T_{O} to colder T_{A}, collapsing to point B for implantation, then heating to body temperature. In some instances, e.g. stents, expanding balloons are used to bring the stent to the desired large-volume state in situ. There is seldom consideration of what may be needed to re-collapse the medical device for explantation. “For example, alloys which are intended to be superelastic at room temperature are generally produced with their active A_{F} temperatures just below room temperature, say in the range of 0-20° C. Such a material will also exhibit good superelastic properties at body temperature (37° C.).” D. Kapoor. Nitinol for Medical Applications: A Brief Introduction to the Properties and Processing of Nickel Titanium Shape Memory Alloys and their Use in Stents. Johnson Matthey Technol. Rev., 2017, 61, (1), 66-76.

It is currently not frequent to consider the process of explantation of expanded nitinol cardiovascular devices, such stents, valves, or heart-assist pumps. As cardiovascular valves are increasingly installed in younger patients, there have been a few cases or explantation reported. In one case, a valve was cooled with water for minimally-invasive removal. Other cases describe sternotomy and surgical explantation. As the field evolves, there will be increased demand to explant valves, or stents, and miniature cardiovascular heart-assist pumps intended for temporary use (temporary Mechanical Circulatory Support Devices, or temporary MCSD). For these explantation cases to be performed with minimally-invasive procedures, the implanted device needs to be collapsed to small-volume shape at 37 deg C. This body temperature is above the atmospheric temperature T_{O} at which the device entered the operating room. The Upper Stress Plateau (USP) and Lower Stress Plateau (LSP) levels have been displaced upwards as illustrated in FIG. 7A, making the device stiffer for explantation at body temperature (about 37 deg C.) than it was at implantation temperature (about 20 deg C.). The methods described herein consider this explantation process at the higher (body) temperature.

While the process is described herein using the example of collapsing the blades of and axial turbomachine pump, the process can be used in all other cases of explantation of cardiovascular prosthetic devices, and all other biomedical devices that need collapsing for explantation, or need collapsing at two or more different temperatures. While the process is described below using the example of collapsing the blades of and axial turbomachine pump, the process can be used in all other industrial cases of shape memory alloys that need to be collapsed at two or more different temperatures. In some applications, devices may need to just change shape, not just fully collapse and expand, at more than 2 different temperatures. In aerospace and control applications, the shape of the device may need to be continuously variable as a function of changing temperature.

FIG. 9 illustrates the stress-strain-temperature curve for shape memory alloys. FIG. 9 can explain the method for removal of biomedical devices at body temperature disclosed herein. Note the material may or may not always recover its zero-strain or large shape, but what breaks the material is when the material reaches fracture strains, illustrated as points X in FIG. 9. Note the breaking strain at temperatures above T_{Md} is less than the breaking strain at body temperature T₈, which is less than the breaking strain at room temperature T₅, which is less than the breaking strain at the colder temperature T₂. The device may be designed to fully recover its original shape when explanted at body temperature starting from point 9. The device may be elastic, as defined herein. The stress-strain-temperature curve above Md is shown in teal in FIG. 9. In the case of a temporary MCSD device, when folded in the catheter at body temperature T₈ (points 9 and above), it must not reach the fracture strain X at temperature T₈.

The method can be described in stress-strain-temperature terms. FIG. 9 (left) illustrates a three dimensional view of the stress-strain-temperature curve of a typical shape memory alloy, FIG. 9 (left) can explain the requirements and method of implantation, use and removal or collapsible shape memory alloy from the human body. FIG. 9 (right) illustrates the two dimensional stress-strain curves at the different temperatures. FIG. 9 (right) can be compared to FIG. 7. There are differences in fracture strain (points X) and corresponding fracture stress, as a function of temperature. The differences in fracture strain may exhibit different variations than those in FIG. 9, see for instance FIG. 7A.

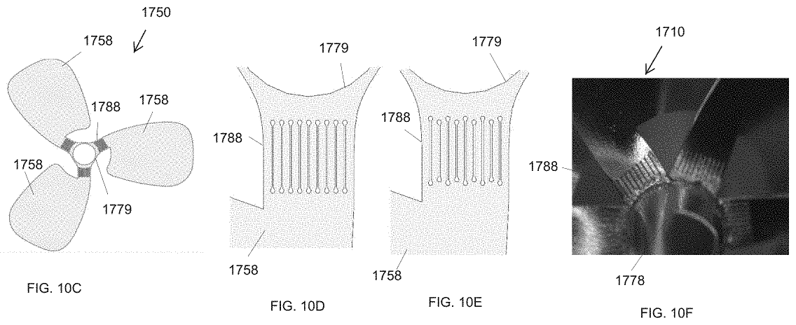

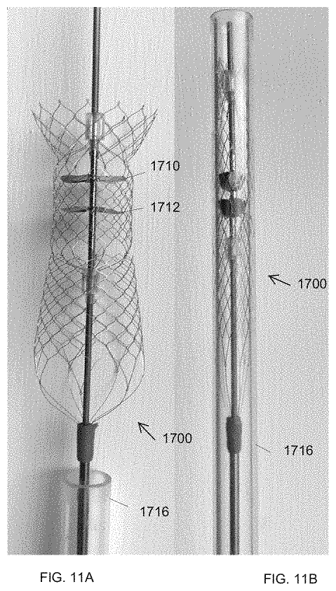

FIG. 10A-10F illustrates a device 1700. The device 1700 can include a miniature heart-assist pump with axial turbomachine blades installed in a stent frame. The device 1700 can include contra-rotating impellers 1710, 1712. The device 1700 can be collapsed and implanted in a collapsed state. The device 1700 can be expanded inside the vasculature at body temperature. The device 1700 can be operated for a period of time at body temperature. The device 1700 can then be collapsed again at body temperature without fracturing, in order to be safely explanted from the human body. FIG. 10A illustrates the device 1700. FIG. 10B illustrates the device 1700 which includes impellers 1710 and 1712 that collapse by folding upstream. FIG. 10C illustrates the impeller portion, also called impeller segment 1750 including three blades 1758 connected by the flat-plate circle 1779 at the center. FIG. 10D illustrates a portion of the blade 1758. FIG. 10E illustrates a portion of the blade 1758. FIG. 10F illustrates a portion of the impeller 1710, composed of two identical impeller portions 1750 in which one is rotated azimuthally 60 degrees from the other, and connected together to each other and to impeller hubs 1778 to form a 6-bladed impeller assembly. FIGS. 11A-11B illustrate the device 1700. The device 1700 can be collapsed within a catheter 1716.

Without loss of generality, as a representative example, the method is described in relation to using the folding blades of a miniature heart assist pump with axial turbomachine blades secured in a stent-like hourglass shape, illustrated in FIGS. 10A-10B. The collapsing is illustrated in FIG. 11A-11B. In some embodiments, each impeller includes six blades. There can be two impeller portions 1750, and each airfoil can include any number of blades. There can be three blades per impeller portion 1750. The impeller portions 1750 can be stacked. The impeller portions 1750 can be stacked to form the impeller 1710. The impeller portions 1750 can be offset. The blades of the impeller portions 1750 can alternate. The blades of the impeller portions 1750 can overlap. The impeller 1710 can provide for blade overlap at the hub circumference. The impeller 1710 can provide for blade overlap at the tip circumference. The impeller 1710 can provide for blade overlap at the hub circumference and the tip circumference. The design can allow for smooth folding of the blades. The design can allow for easier folding compared with three dimensional blade shapes (in which the blade thickness varies from leading to trailing edge and from hub to tip). The design can allow for stacked impeller portions 1750. The design can allow for an overlap between blades at a hub. The design can allow for a large number of blades per impeller 1710. The larger number of blades per impeller 1710 can decrease the blade-to-blade flow gap. The larger number of blades per impeller 1710 can increase the solidity, i.e. more blades of the same shape in the circumference. The larger number of blades per impeller 1710 can provide better guidance to the flow. The larger number of blades per impeller 1710 can provide higher hydrodynamic efficiency.

The blades can be manufactured from sheets of material. The three blade impeller portion shape 1750 can be cut out of sheets of shape-memory alloy. A pair of these impeller portions 1750 can be placed together. The impeller portions 1750 can be rotated azimuthally 60 degrees. The impeller portions 1750 can be connected to two cylindrical half shafts or hubs 1778, one upstream and one downstream of the blades. These shafts can be considered the upstream hub and the downstream hub. The flat-plate circles 1779 of impeller portions 1750 and the hubs 1778 can be welded together to form the impeller 1710. The flat-plate circles 1779 of impeller portions 1750 and the hubs 1778 can be connected together by adhesive, glue, fasteners, weld, and other means. The impeller portions 1750 and the hubs 1778 can be heat treated to achieve the three dimensional flat-plate blade shape. Additional features of the device 1700 are described in a nonprovisional utility patent application entitled COLLAPSING MECHANICAL CIRCULATORY SUPPORT DEVICE FOR TEMPORARY USE, filed as on the same day herewith, and U.S. Provisional Application No. 63/279,826, filed Nov. 16, 2021, which are incorporated by reference herein in their entirety. The hubs 1778 may also be secured azimuthally without welds with methods described in these related applications. For instance, the hubs 1778 may be secured by fixing the location with indexing components and avoiding the weld. In some embodiments, the weld weakens the structure and alters the metal properties. The transformation from austenite to martensite can be less predictable after weld. The indexing mechanism addresses this issue by avoiding welds.

In device 1700 the hydrodynamic efficiency of the pump is determined by the amount of regurgitant flow (backflow), from higher pressure downstream, to lower pressure upstream, occurs at the gap between the impeller tip and the waist. Flow shear in the same gap causes hemolysis, the size of the gap determining to a large degree the level of hemolysis of the pump. It is crucial to control this gap using processes described in FIG. 7B. This can be done one of two ways: after one cycle of folding and unfolding the waist, or after a series of cycles of folding and unfolding the waist, resulting in nitinol “training”, described in the above. These processes can be used to prescribe the specific size of the inner diameter of the waist, thus controlling the impeller tip to waist inner diameter. In turn, this can be used to balance considerations between hydraulic efficiency and hemolysis. In some embodiments, one cycle is used to get to one desired size of the waist inner diameter. In some embodiments, a series of cycles is used to reach the desired size of inner diameter of the waist. In device 1700, the most strained component is the segment 1788 of thin material connecting one blade 1758 to its hub 1778. The segment 1788 forms a hub-to-blade segment that connects the blade 1758 to flat-plate circle 1779 and via that to a hub 1778. The impeller portion 1750 can include a central opening. The segments 1788 can extend from the central opening. The segments 1788 can be any elongate shape. The segment 1788 can be configured to fold. The segment 1788 can form the base of the blade 1758. This segment 1788 can facilitate folding, as described herein. If this device 1700 breaks on collapsing, the segment 1788 is where the device 1700 will likely break first. The device could break elsewhere too, and the processes described herein can be applied to the most stressed component. The device 1700 can be designed so that it does not break at representative temperature T₈. In the case of removable heart assist pump, the device 1700 can be designed so that it does not break at representative temperature T₈ when collapsed into the catheter at body temperature, so that it can be explanted intact, in this instance without leaving broken blades in the vasculature. Without loss of generality, the concepts described can be applied to other medical devices, and other industries when shape memory alloys must be collapsed intact at different temperatures. The device 1700 can be designed so it does not reach fracture point as it folds and unfolds at Temperature T8. The device 1700 can be designed so it remains in the elastic regime as it folds and unfolds at temperature T8. The segment 1788 can be called the blade hub interconnect or hub-blade connector.

The device 1700 can include a miniature heart-assist pump. The device 1700 is brought to the operating room at room temperature. The device 1700 is expanded in its geometry as shown FIGS. 10A and 11A. This shape can be considered the large-volume, zero-stress, zero-strain shape. The room temperature can be at about 20° C. This corresponds to point 1 at 20° C. in FIG. 9. At that point 1, the blades are in austenitic state, characterized by the red dotted stress-strain pseudo-elasticity loop.

Subsequently, the device 1700 can be inserted in an ice bath. The device 1700 reaches in its expanded geometry, the large-volume zero stress zero-strain shape shown at the FIGS. 10A and 11A. The device 1700 reaches in its expanded geometry, the large-volume zero stress zero-strain shape at a cooler temperature than room temperature. The device 1700 reaches in its expanded geometry, the large-volume zero stress zero-strain shape at point 2 on FIG. 9. This point 2 corresponds to a temperature at about 0° C. in FIG. 9. The temperature T₂ at point 2 is lower than room temperature T₁ at point 1. The temperature T₂ may be lower than 0° C. if a cooling spray is used. At that temperature T₂, the stress-strain curve of the device 1700 is the green curve in FIG. 9. The Upper Stress Plateau (USP) of the green curve is at lower stress than the Upper Stress Plateau (USP) of the red curve. The Upper Stress Plateau (USP) of at lower temperature, such as in an ice bath, is at lower stress than the Upper Stress Plateau (USP) of a higher temperature, such as room temperature. This means that the device 1700, including the connecting member 1788 between the blade 1758 and the hub 1778, is softer at the cooler temperature T₂ than it was at room temperature T₁. The device 1700 is easier to collapse at the cooler temperature T₂ than at room temperature T₁. Therefore, at the cooler temperature T₂, the device 1700 can be collapsed into a constraining shape, such as within a catheter 1716. FIG. 11B illustrates the collapsed shape of the device 1700 within the catheter 1716.

In the process of collapsing the device 1700 at cooler temperature T₂, the stress and strain may reach up to any point in the elastic region between Point 2, Point 3a, Point 3, and Point 3b in FIG. 9 along the green curve. The point reached depends on the stress level. In theory, the device 1700 can also reach points higher than Point 3b, provided the stress stays in the elastic regime below the Point X. The device 1700 can reach any point in the elastic regime, so that if the device 1700 is fully unloaded at this cooler temperature (T₂), the device 1700 will reach Point 4. At Point 4, the device is with permanent deformation and positive strain. In most instances, the device 1700 will be loaded up to Point 3 between Point 3a and Point 3b. In most methods, the device 1700 is not unloaded from the catheter 1716 at this temperature T₂, and thus the device 1700 never reaches Point 4. This is in contrast to the theoretical descriptions of the process in reference textbooks and papers. The example of catheter is for illustration purposes, and any shape-constraining device may be used, especially in different industries. The ice bath is for illustration purposes, and several alternative ways to cool the device 1700 to T₂ may be employed. Point 4 is the point with permanent deformation and positive strain. Point 4 is at zero stress, positive strain. Point 3a and up to maximum point 3b are points that avoid permanent plastic deformation.

At this point, conventional teachings suggest that the shape memory alloy is reheated to a higher temperature, such as room temperature T₁. The reheating can reach Point 6 between Point 6a and Point 6b on the red line if constrained. The reheating can reach Point 5 at zero stress and zero strain if unconstrained. The reheating can cause the device 1700 to recover the initial large-volume state at Point 5. Conventional teaching describe how the device goes from Point 3 to Point 4, and then from Point 4 at zero stress along the green dotted line to Point 5.

It is important to recognize that this does not happen to cardiovascular devices implanted in the human body. Instead, starting from Point 3 between Point 3a and Point 3b, or any point on the elastic regime along this green line, subsequently the device 1700 is inserted in the human body, at the small-volume, positive stress and positive strain state. The device can be represented in our example by the shape shown in FIG. 11B. The human body is at temperature about 36.7 deg C. The cooler device (it started from Points 3 at T₂) absorbs thermal energy from the body, and is heated to temperature T₈, which is not on the red line as described in conventional teaching, but on the fuchsia line in FIG. 9.

The implanted device 1700 reaches the fuchsia line FIG. 9. The Upper Stress Plateau (USP) and Lower Stress Plateau (LSP) shown in fuchsia corresponding to the higher temperature T₈ are higher than the corresponding Upper Stress Plateau (USP) and Lower Stress Plateau (LSP) shown in red corresponding to room-temperature T₁. The corresponding fracture strains are at different levels for the fuchsia line and the red line (FIG. 9, bottom right). This means that if the device 1700 is still loaded in the catheter then it is along Point 7 between Point 7a and Point 7b when it reaches the higher temperature T₈. It takes more force to unload the device 1700 from the catheter 1716 at T₈ than it would take to load the device 1700 in the catheter 1716 at T₁ and at T₂. The device 1700 is and feels stiffer at higher temperature T₁ than at cooler temperature T₂ because of the relative location of Upper Stress Plateau (USP). The device 1700 is and feels stiffer at higher temperature T₈ than at temperature T₂ because of the relative location of Upper Stress Plateau (USP). The device 1700 is and feels stiffer at higher temperature T₈ than at cooler temperature T₁ because of the relative location of Upper Stress Plateau (USP). It takes more force to unload it from the catheter at temperature T₈ than at T₁. For the device 1700 to not experience fracture, the designer must ensure that it stays below fracture strain at T₈ corresponding to Point X. The device may need to stay within elastic deformation regime below 7b=9b in some applications. In applications in other industries, temperature T₈ may be anywhere on the temperature scale shown in FIG. 9. Thus, this introduces additional considerations for making the device change shape at a different temperature than T₁ and T₂, for temperatures anywhere on the temperature axis, while staying in the elastic regime, or without reaching fracture point at this third temperature.

Upon being implanted, the device 1700 reaches temperature T₈ while loaded via the catheter 1716 inside the body of the patient. Contrary to conventional teachings, if the device 1700 reaches body temperature T₈ while inside the catheter 1716, then the device 1700 still has deformation (positive strain), and therefore it moves from Point 3 at temperature T₂ to Point 7 at temperature T₈. It is important to design the shape of the deforming device 1700 so that stresses and strains for temperature T₈ are anywhere along Point 7a to Point 7 to Point 7b, below fracture at Point X, or in elastic regime if that is the target, as in the case of the heart pump. When the device 1700 is unloaded from the catheter 1716 in the body of the patient, the device will reach its original large-volume state (shown in FIG. 11A) with zero strain when it is at zero stress at Point 8. In theory, the device 1700 can also reach points higher than Points 7b, provided it stays in the elastic regime so that if fully unloaded at this temperature (T₈) it will reach Point 8 (large volume, zero stress, zero strain state, identical in shape to the state on FIG. 11A). For devices that must not fracture, the device 1700 must stay below fracture strain at temperature T₈.

Depending on how rapidly the device 1700 is installed in the body of the patient and unloaded from the catheter 1716, and how rapidly the device 1700 is heated from temperature T₂ to T₈, it is also possible that the device follows any path on the three dimensional stress-strain temperature curve from Point 3 (where it is in the low-volume configuration illustrated on the FIG. 11B) to Point 8 (where it is in the large-volume configuration illustrated on the FIG. 11A). It is also possible that the device follows any path on the three dimensional stress-strain temperature curve from Point 3 (where it is in the low-volume configuration illustrated on the FIG. 11B) to Point 8 (where it is in the large-volume configuration illustrated on the FIG. 11A), or at any point from 8 to 8a, 8b, 9a, 9b, and below X. It is noted that stents may be implanted compressed at positive stress-strain territory at T₈ pressing against the blood vessel wall.

Most such devices are not usually explanted, e.g. by the reverse procedure from implantation. In the case of the device 1700, the pump can operate at some rpm inside the body for a period of time. At the end of this period of time, the pump must be explanted from the body without breaking. This removal is harder to do at body temperature T₈ than the loading process at T₂, because the Upper Stress Plateau (USP) curves at temperature T₈ are higher than at temperature T₁ and which are higher than temperature T₂, and the breaking stresses at these temperatures are also different. Similarly, for the device not to fracture, while collapsing at temperature T₈, it must stay below the corresponding fracture strain at Point X.

In order to explant the device, the device 1700 at temperature T₈ is collapsed into a catheter 1716, and then removed. In the vast majority of cases, it is not feasible to cool the device 1700 to temperature T₁ or temperature T₂ inside the human body, in order to follow the reverse process of implantation for explantation. It is therefore required to collapse the device at temperature T₈ inside a catheter to Point 9, between Points 9a and Point 9b, in FIG. 9. In theory, it is acceptable to collapse the device 1700 to any positive strain below the breaking strain at Point X at T₈ on the fuchsia line. Provided the device 1700 does not fracture inside the body of the patient, the device 1700 can be collapsed inside the catheter 1716, and then explanted. After the device 1700 is explanted, the device 1700 will be further cooled from body temperature T₈ to room temperature T₁. When it is unloaded from the catheter at T₁, if the device 1700 has remained in the elastic regime throughout, the device 1700 will return to Point 10, in the shape illustrated in FIG. 11A, following the Lower Stress Plateau (LSP) unloading curve along the red line. If it has reached into plastic deformation during the explantation process, then the device will reach temperature T₁₀ at zero stress and some positive strain.

The teal line corresponds to temperatures higher than the martensite deformation temperature Ma in FIG. 9. The fracture stress and strain values (point X) at alloy temperatures higher than the martensite deformation temperature Ma in FIG. 9 are lower than those at temperature T₈ in FIG. 7. For most current alloys used in medical devices, martensite deformation temperature Ma is higher than body temperature T₈. However, it may be possible to apply the above implantation and removal techniques for alloys where martensite deformation temperature Ma less than temperature T₈, provided the stresses and strains are low enough for the other conditions for shape recovery. It may be possible to apply the described processes to other industries. It would be obvious to apply the concepts disclosed hereto other industries and in cases where the 3 or more temperatures where the structure changes shape at more than 2 different temperatures T₁, T₂, T₈ etc., where each of the 3 temperatures may be the middle temperature between the other two.

Therefore, it is important to recognize that the method of design of such device 1700 for explantation is at the higher Upper Stress Plateau (USP) curves of temperature T₈ (fuchsia line), and not those at temperature T₅ or T₁ (red line) or T₂ (green line). Therefore, it is important to consider the corresponding breaking strain levels. It is important to consider the corresponding breaking strain levels at temperature T₈ (fuchsia line) which is lower than the breaking strain levels at T₅ or T₁ (red line) or T₂ (green line). The methods of calculating the stresses, strains and forces for the design of these devices 1700 may be numerical (e.g. Finite Element Methods (FEM) programs allow modelling the Upper Stress Plateau (USP) and Lower Stress Plateau (LSP) in the calculations), or theoretical, or experimental. Some examples are provided herein.

With respect to the shape memory alloy folding and unfolding methods described herein, in this example, the likely most critical component of the temporary-use explantable device 1700 is the segment 1788 connecting the impeller portion shape 1750 to the flat plate circle 1779 and the hub 1778. This segment 1788 is a small flat horizontal plate, connecting the hub 1778 via the flat-plate circle 1779 to the three dimensional impeller portion shape 1750 of the blade 1758. This flat plate of the segment 1788 must be stiff enough to minimize blade deflection. The blade 1758 is subjected to forces that the blade 1758 experiences upstream from the fluid pressure that the blade 1758 generates. The blade 1758 is subjected to forces by the action-reaction principle. The impeller 1710 must reasonably maintain its shape under these forces. The blade 1758 must not deflect too far upstream under these forces. The flat plate of the segment 1788 needs to be stiff enough so it does not deflect with this force, yet flexible enough to allow the blade 1758 to fold upstream into the catheter as shown in FIGS. 11A-11B. The flat plate of the segment 1788 also needs to be made so that the stresses on of the segment 1788 do not exceed the plastic-deformation levels at loading temperature T₂, and breaking stress at body temperature T₈ for safe explantation.

One way to meet the conflicting requirements is to introduce slits into this vertical plate of the segment 1788 as shown in the middle part of FIGS. 10C-10F. The force, deflection and stress calculations can be made theoretically, or numerically, or by trial and error.

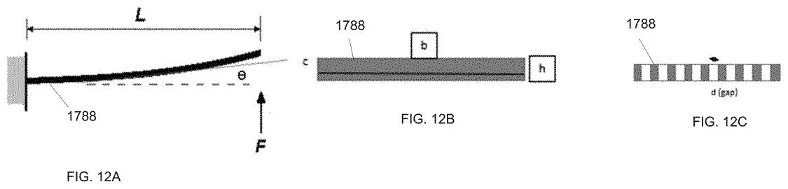

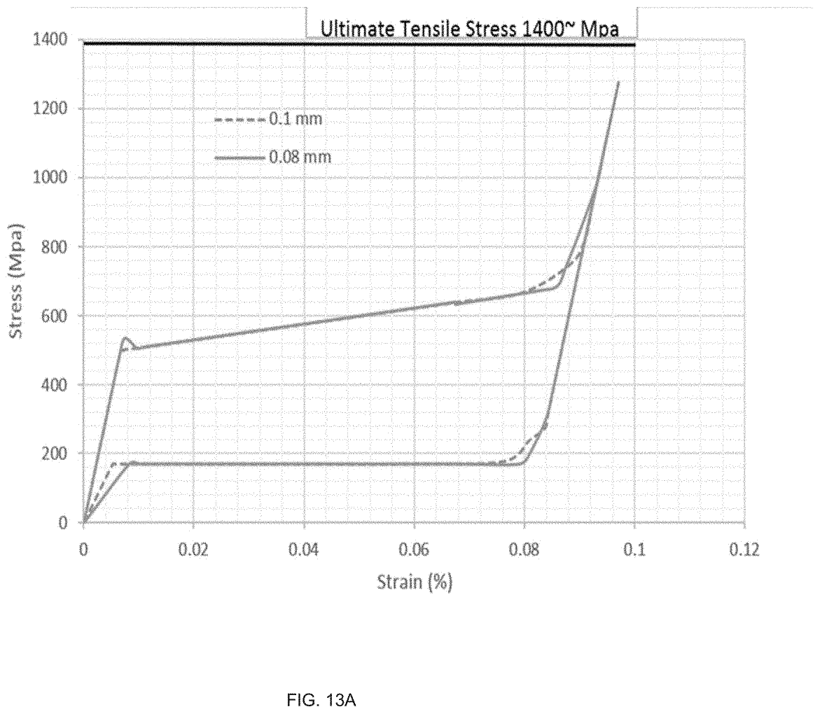

FIG. 12A illustrates the segment 1788 with a length and a force applied. The maximum deflection angle can be determined by Equation A. The maximum stress can be determined from Equation B. Combining these equations can provide Equation C. The maximum deflection angle can be a function of the maximum stress. In the equations below, I: Moment of inertia; E: Module of elasticity; L: Distance from the Applied force to fixed end; F: Applied Force by the catheter; and c: Distance from the neutral axis to the extreme surface.

$${Θ}_{max} = \frac{F \times {L}^{2}}{2 EI} \qquad Equation A$$

$${σ}_{max} = \frac{F \times L \times c}{I} \qquad Equation B$$

$${Θ}_{max} = {σ}_{max} ( \frac{L}{2 E c} ) \qquad Equation C$$

From Equation C, for a given deflection angle (Θₘₐₓ), of a given material (E), in order to reduce the maximum stress, either the length (L) must increase (and therefore also the catheter diameter), or the flat-plate thickness (c) must decrease. The moment of inertia (I), including the effect of geometrical stiffness would not have any impact on the maximum stress.

FIG. 12B illustrates the solid plate cross-section. In the equations below, I: Moment of inertia; b: is plate width; and h=2c: is plate height.

$$I = \frac{1}{12} b {h}^{3} \qquad Equation D$$

FIG. 12C illustrates the slitted plate cross-section. The plate has the same dimensions at FIG. 12A shown in FIG. 10, but with eight slits in the cross-section. Thus, by adding slits, the moment of inertia decreases. In the equation below, k is the number of slits where material has been cut, and k equals 9 in FIG. 10 and k equals 8 in FIG. 12C.

$$I = \frac{1}{12} b {h}^{3} - k \frac{1}{12} d {h}^{3} \qquad Equation E$$

According to the equations above, for a given deflection angle, the reduced moment of inertia by adding one or more slits leads to a reduced force, but does not have any effect on the stress level.

Maximum surface stress is affected by plate thickness of the segment 1788. To reduce maximum surface stress, the plate thickness of the segment 1788 can be reduced. By adding slits to the segment 1788, this reduces the force to bend the blades, but does not affect stress on the material surface. The slits may be placed closer to the hub, or closer to the blade, or be made in a variety of configurations such as those shown in FIGS. 10D-10E, to encourage bending at a specific point along the flat plate of the segment 1788. In some embodiments, stress-relieving circles or other similar shapes may be cut into the ends of the slits, as illustrated in FIGS. 10D-10E.

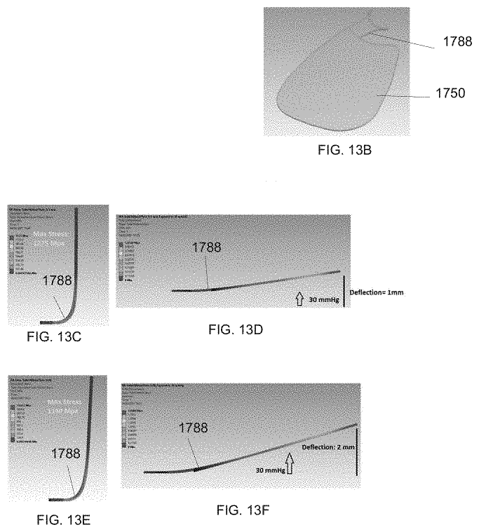

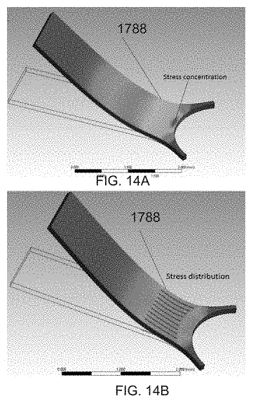

FIGS. 13A-13F are a representation of the concepts presented in this method using computations, such as Finite Element Method (FEM) computations using ANSYS. FIG. 13A shows the mathematical model used for the Upper Stress Plateau (USP) (about 600 kPa) and Lower Stress Plateau (LSP) (about 180 kPa) of shape memory alloy in the martensite-austenite transformation regime and the 1,400 MPa stress below which the material is elastic and above which plastic deformation forms. FIG. 13B illustrates the impeller portion shape 1750 simulated as a two-dimensional shape and the supporting flat plate of the segment 1788 securing the blade 1758 to the circular hub 1778. FIGS. 13C-13D illustrate the flat plate of the segment 1788 having a thickness of 0.1 mm. This shows the simulated blade stress on plate thickness of 0.1 mm when folded in a 6 mm flat catheter and deflection when subjected to the force from 30 mm Hg pressure rise in the pump. FIGS. 13E-13F illustrate the flat plate of the segment 1788 having a thickness of 0.08 mm. This shows the simulated blade stress on plate thickness of 0.08 mm when folded in a 6 mm flat catheter and deflection when subjected to the force from 30 mm Hg pressure rise in the pump.

Computations for flat-plate supporting structure 1788 and blade thickness of 0.1 mm and 0.08 mm are compared, without considering the effect of the slits. We consider the effect of plate thickness on deflection for a given force on the blade (caused by 30 mm Hg pressure rise), and surface stress for folding the blade (modelled as a flat plate) into a 6 mm restriction (where the catheter is also modelled as infinite flat plate into the paper).

The computed results verify the theoretical calculations above. Among the folded plates the thicker 0.1 mm plate exhibits maximum stress 1,275 MPa at the plate bend, and the thinner 0.08 mm plate exhibits lower maximum stress 1,140 MPa at the same point. Both of these values are above the Upper Stress Plateau (USP), but in the elastic regime for the shape memory alloy stress-strain-temperature relation, and below the ultimate tensile stress of 1,400 MPa. Correspondingly, when subjected to the same upward force generated from a pump pressure rise of 30 mmHg, the thinner blade of 0.08 mm deflects about 2 mm upstream, and the thicker blade of 0.1 mm deflects 1 mm upstream. The computed results assist in determining acceptable compromises between undesirable blade deflection and stress levels, so the shape memory alloy does not enter into plastic deformation when loaded into the catheter at T₂ for implantation, and does not fracture when re-loaded into the catheter at T₈ for explantation. The 0.1 mm folded blade reaches 1275 MPa. This is higher than the folded 0.08 mm blade, which reaches 1140 MPa. As a thicker plate is used to minimize deflection during unfolded 30 mmHg operation, a stiffness can be reached in excess of 1400 MPa at the folded blade condition, beyond which plastic deformation will form. For even thicker plates reaching even higher stress-strain points in the folded blade condition, the strain fracture can be reached at point X.

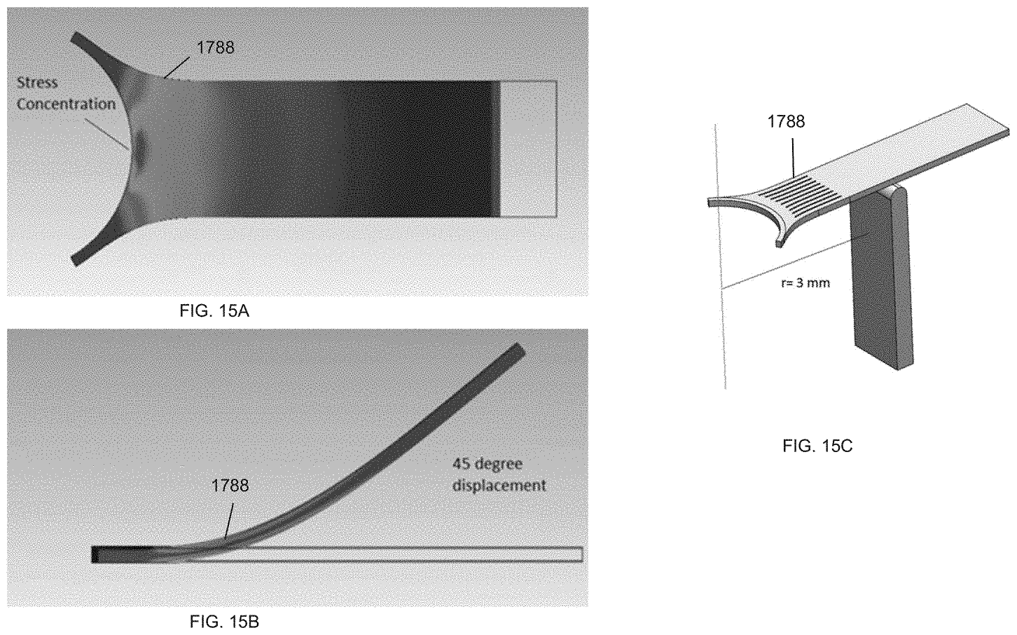

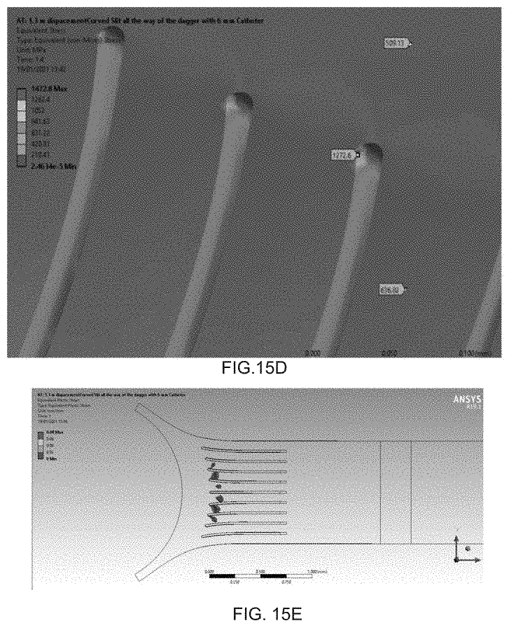

FIGS. 14A-14B illustrate how slits along the flat plate of segment 1788 lower the maximum stress and redistribute the stresses along the segment 1788. FIG. 14A illustrates the stress concentration without slits. FIG. 15B illustrates the stress concentration with slits. These figures illustrate with FEM computations how the introduction of slits can be used to reduce the maximum stress at the folding flat plate surfaces, thus allowing folding the shape memory alloy for implantation and explantation with lower stresses

FIGS. 15A-15E illustrate of simulation of surface stresses for a 0.3 mm plate of segment 1788 without slits (maximum stress at the hub), and FIG. 15C-15E illustrate the formation of plastic deformation at the sharp points of the slits when bent as shown in the figure. FIGS. 15B and 15E have slits. FIG. 15A illustrates the stress concentration. FIG. 15B illustrates the 45 degree displacement. FIG. 15C illustrates the segment 1788 with slits. FIG. 15D illustrates the location of the maximum stress. FIG. 15E illustrates maximum strain. These figures illustrate Finite Element Method (FEM) under various models for a 0.3 mm plate, identifying the locations of maximum stress and strain locations where the stress indicates plastic deformation. The computations in combination with the above considerations indicate that the flat plate needs to be thinner in order to stay in the elastic deformation regime. Alternatively, similar results would be obtained if the flat plate was thinner, but the Upper Stress Plateau (USP), and ultimate tensile stress levels were lower than those shown in FIG. 14. For a 0.3 mm plate, the force is applied distance r=3 mm from the axis. In some embodiments, the plate is thinner, either 0.1 or 0.08 mm.

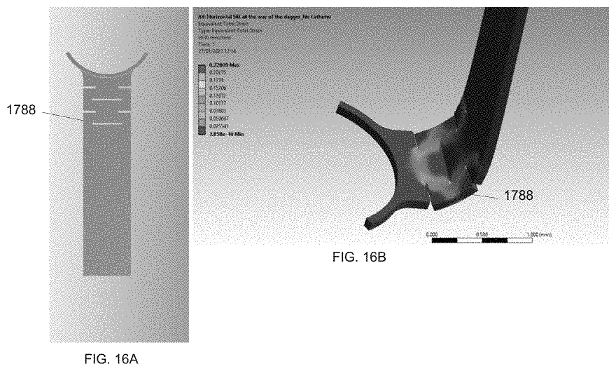

FIGS. 16A-16B illustrate alternative flat-plate configurations of the segment 1788 to minimize stress at the maximum bend point so the folded shape memory alloy plate stays in the elastic regime at both temperature T₂ and temperature T₈. Alternative shapes to minimize stresses at the supporting plate are shown. FIG. 16A illustrates one or more slits. The slits can extend from the edges of the segment 1788. The slits can extend through a middle portion of the segment 1788. The slits can be axially aligned. The slits can be laterally displaced. The segment can include horizontal slits along a portion of the length.

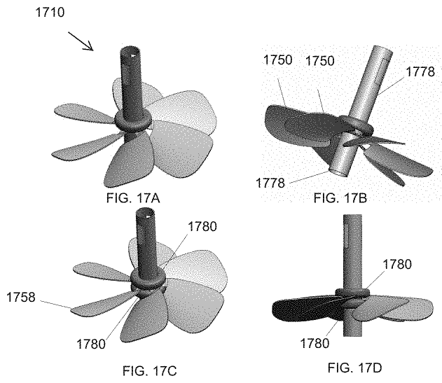

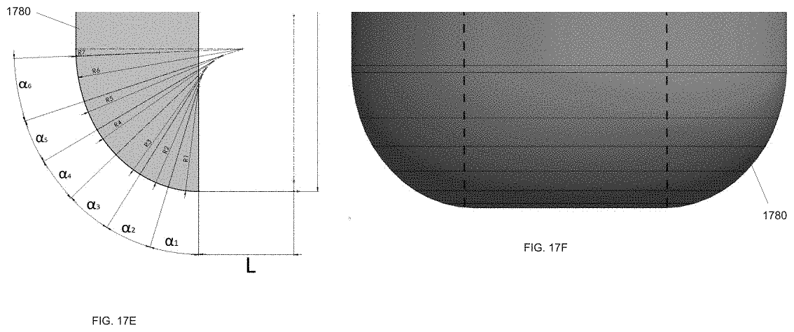

FIGS. 17A-17D illustrate the placement of supporting structures upstream or downstream of the flat plate supporting the blade, to facilitate bending of the flat plate at the (desired) shape, in this instance a circle, thus minimizing stresses at the folding region of the shape memory alloy. The impeller 1710 is shown. The impeller 1710 can include the upstream and downstream hubs 1778. The hubs 1778 can support the blades. The hubs 1778 can support the impeller portions 1750 containing the blades. The impeller 1710 can include one or more supporting structures 1780. The placement of supporting structure 1780 can be upstream of the impeller portion 1750. The placement of supporting structure 1780 can be downstream of the impeller portion 1750. The supporting structure can be placed upstream of the blades, if the blades were to be folded upstream. The supporting structure can be placed downstream of the blades, if the blades were to be folded downstream. In the illustrated embodiment, the supporting structures 1780 include an upstream and downstream supporting structures 1780. The supporting structures 1780 can be positioned relative to the impeller portion 1750 that supports the blades. The supporting structures 1780 can be positioned relative the hubs 1778. The supporting structures 1780 can be upstream and downstream of the segment 1788. The supporting structures 1780 can facilitate bending of the impeller portion 1750 at the desired shaped. Bending segment 1788 at constant radius in this instance, distributing stresses evenly along the circle, rather than uncontrolled bending that could be of higher curvature (lower radius) at any location along the bending region. Other supporting shapes can also be used to control this bending rate along segment 1788. The supporting structures 1780 can facilitate bending of the impeller portion 1750, without breaking at 1788, to collapse the impeller 1710. The supporting structures 1780 can minimize stresses at the folding point of the segment 1788.

The impeller 1710 can include one or more supporting structures 1780 near the hub 1778 of the blades. The supporting structures 1780 can improve shaft rigidity near the blades. The supporting structures 1780 can eliminate or reduce the slow-flow regions near the blades. The supporting structures 1780 can improve hydrodynamic performance. The contra-rotating blades 1710 can be the result of improved manufacturing process for the folding blades. Each impeller can include two, three, four, five, six blades or any range of blades. Each impeller can have shaped blades. The number and shape of blades can facilitate smooth folding. The blades 1710, 1712 are made from flat plates formed into impeller portions 1750. The blades 1710, 1712 are shaped into three-dimensional objects with varying blade angle from hub to tip. The supporting structures 1780 can be o-ring shapes or similar shapes. The supporting structures 1780 can be configured to eliminate slow flow regions near the hubs 1778.

The curvature controller of the supporting structures 1780 may have varying radius distribution as shown in FIGS. 17E and 17F. This varying radius distribution can accommodate stress or strain levels below a desired point along the length of the blade-hub interconnect or segment 1788. The curvature controller of the supporting structures 1780 has varying radius R at different angles alpha. In some embodiments, the blade-hub interconnect or segment 1788 may be a 3D shape, not 2D and flat. This is in order to accommodate the transition from the hub to the stagger angle of the blade at the hub shown in FIG. 13B. This means that the blade-hub interconnect or segment 1788 during folding is subjected to bending stress along the impeller axis and in addition torsional stress twisting the blade-hub interconnect or segment 1788 as it bends to fold. This subjects the blade-hub interconnect or segment 1788 to the combination of bending and torsion stress and therefore bending and torsional strain, both of which must be accounted in order to have the folder device below the targeted strain, or below the targeted stress. The changing radius of the curvature controller shown in FIG. 17e can be used to control the rate of distribution of stress, or strain, along the 3D bending shape of the blade-hub interconnect or segment 1788, in order to keep the combined bending and torsional stresses below a target level; or the resultant strain below a target level for deformation control.

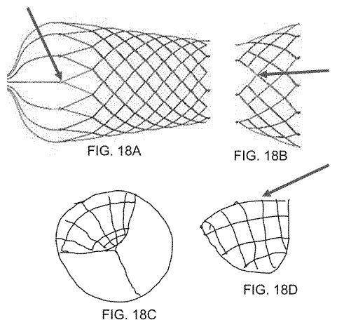

FIGS. 18A-18D illustrate the places where the methods described herein may be applied to shape memory alloy stents, or to shape memory alloy heart valves. FIGS. 18A-18B shows samples of stent frames. FIGS. 18C-18D illustrate a tri-leaflet valve where the shape memory alloy is the supporting shape memory alloy structure, shown in one leaflet. The heart valve shape shown in the figures may be inserted and anchored in another receiving shape memory alloy shape placed in the location of the native valve. The shape memory alloy collapsing method at T₁, T₂, T₈ etc. described herein may be applied to high-stress and high strain points to the perimeter of the stent frames on one leaflet for collapsing the device, or to the perimeter of the valve for collapsing it in a catheter, or to the supporting receiving shape memory alloy in which the valve is inserted mentioned above, and/or on one leaflet of the valve. The shape memory alloy supporting structure of the stent or the valve may be covered with a biocompatible material or with thin layers of nitinol sheets. In some embodiments, Nitinol subframes for valve leaflets in FIG. 18D are put together to form a bi-leaflet or tri-leaflet valve subframe of FIG. 18C, which is collapsed with the methods described herein, inside a surrounding cylindrical Nitinol subframe such as that shown in FIG. 18B. The device of FIG. 18B is implanted first and forms the perimeter of the valve. The device of FIG. 18B may be made for permanent implantation or be removable from that location. The device of FIG. 18C is implanted inside the device of FIG. 18B with the method described herein. In some embodiments, the device of FIG. 18C is held inside the device of FIG. 18B by the expanding force in the device of FIG. 18C, or by anchors. This is done so that the device of FIG. 18C may be detached from the device of FIG. 18B after this has endothelialized, with the intention that the device of FIG. 18C is explanted a later date but the device of FIG. 18B stays permanently implanted. The device of FIG. 18C can be anchored inside the device of FIG. 18B, and the device of FIG. 18B may endothelialize, but the device of FIG. 18C may still be removed and replaced with another device of FIG. 18C.

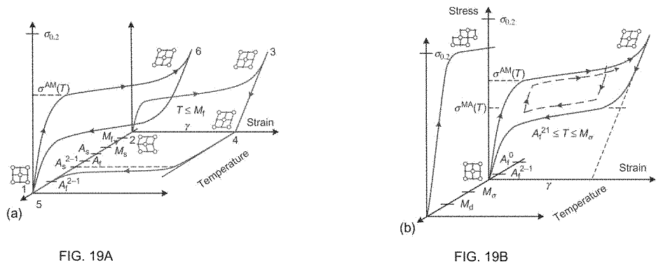

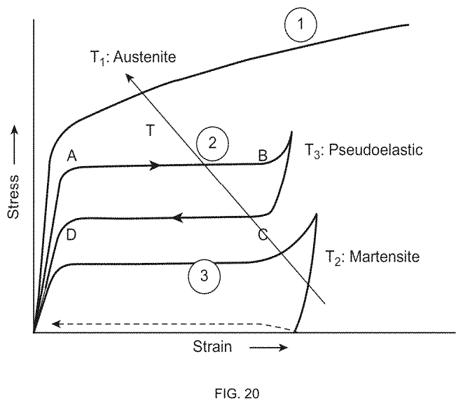

FIGS. 19A-19B illustrates the macroscopic thermomechanical behavior of shape memory alloy materials in stress-strain-temperature coordinates. FIG. 20A illustrates the one-way memory effect. FIG. 20B illustrates pseudoelasticity with internal hysteresis loops and plastic-slip deformation.

FIG. 20 illustrates the stress-strain curves of shape memory alloys at different temperatures.

In some embodiments, a nitinol bending method is provided. The application of the method is described in relation to embodiments of a medical device, but other devices are contemplated. The method can be used with different devices. The method can be used with different industries.

Although the present invention has been described in terms of certain preferred embodiments, it may be incorporated into other embodiments by persons of skill in the art in view of the disclosure herein. The scope of the invention is therefore not intended to be limited by the specific embodiments disclosed herein, but is intended to be defined by the full scope of the following claims. It is understood that this disclosure, in many respects, is only illustrative of the numerous alternative device embodiments of the present invention. Changes may be made in the details, particularly in matters of shape, size, material and arrangement of various device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. The claims below are representative claims, and may be restructured and combined with other features described in the embodiments herein.

1-71. (canceled)

72. A method comprising:
exposing a component comprising a shape memory alloy to a first temperature;
exposing the component comprising the shape memory alloy to a second temperature;
folding the component comprising the shape memory alloy at the second temperature, wherein the second temperature is different than the first temperature;
exposing the component comprising the shape memory alloy to a third temperature;
unfolding the component comprising the shape memory alloy at the third temperature, wherein the third temperature is different than the first temperature and the second temperature; and
folding the component comprising the shape memory alloy at the third temperature,
wherein the component stays below a fracture point at the first temperature, the second temperature, and the third temperature.

73. The method of claim 72, wherein the component comprising the shape memory alloy comprises a component of an implantable cardiovascular support.

74. The method of claim 72, wherein the component comprising the shape memory alloy is used in an aerospace application.

75. The method of claim 72, wherein the first temperature comprises room temperature.

76. The method of claim 72, wherein the second temperature is less than the first temperature.

77. The method of claim 72, wherein exposing the component comprising the shape memory alloy to the second temperature comprises exposing the component comprising the shape memory alloy to an ice bath or a cooling spray.

78. The method of claim 72, wherein the component comprising the shape memory alloy is softer at the second temperature than the first temperature.

79. The method of claim 72, wherein folding the component comprising the shape memory alloy at the second temperature comprises collapsing the component comprising the shape memory alloy into a catheter.

80. The method of claim 79, wherein the component comprising the shape memory alloy is exposed to the third temperature while in the catheter.

81. The method of claim 72, wherein the third temperature is greater than the second temperature.

82. The method of claim 72, wherein the third temperature is greater than the first temperature.

83. The method of claim 72, wherein the third temperature comprises body temperature of a patient.

84. The method of claim 72, wherein the component comprising the shape memory alloy is stiffer at the third temperature than the second temperature.

85. The method of claim 72, wherein the component stays under the fracture strain at the first temperature, the second temperature, and the third temperature.

86. The method of claim 72, wherein the shape memory alloy comprises nitinol.

87. The method of claim 72, wherein the component comprising the shape memory alloy is folded at the third temperature for explantation of the component.

88. The method of claim 72, wherein the component comprising the shape memory alloy is stiffer for explantation than for implantation.

89. The method of claim 72, wherein the component comprising the shape memory alloy comprises a segment connecting two components.

90. The method of claim 72, wherein the component comprising the shape memory alloy comprises slits.

91. The method of claim 72, wherein the component comprising the shape memory alloy is configured to fold against a support structure.
